(19) United States
(12) Patent Application Publication
(10) Pub. No.: US 2013/0060211 A1
(43) Pub. Date: Mar. 7, 2013

(54) CONTINUOUS BLADDER IRRIGATION ALARM

(71) Applicant: John D. Adams, JR., Jackson, MS (US)

(72) Inventor: John D. Adams, JR., Jackson, MS (US)

(21) Appl. No.: 13/601,315

(22) Filed: Aug. 31, 2012

Related U.S. Application Data
(60) Provisional application No. 61/622,328, filed on Apr. 10, 2012.
(60) Provisional application No. 61/529,564, filed on Aug. 31, 2011.

(51) Int. Cl. A61M 1/00

(52) U.S. Cl. 604/327

(57) ABSTRACT

An alarm capable of determining the flow time remaining of irrigation fluid is disclosed. It is also contemplated that the device can detect a partial or complete blockage in the system or empty fluid bag and alert medical personnel locally or remotely as required. The device is capable of calculating the time remaining before the CBI irrigation fluid in the irrigation fluid bag reaches a pre-set level that necessitates changing the bag or the actual time of day or night when the irrigation will be approaching the critical level. Additionally, the user can be alerted to the CBI irrigation fluid in the irrigation fluid bag reaching a pre-set level that necessitates changing the bag before the fluid level becomes critical.

### FIELD OF THE INVENTION

The present invention relates generally to the field of gravity irrigation monitoring devices. More particularly and precisely, the present invention relates to the field of gravity irrigation monitoring devices for the specific procedure of continuous bladder irrigation.

### BACKGROUND OF THE INVENTION

Continuous Bladder Irrigation (CBI) is used primarily following certain surgeries to keep the bladder clear and free of blood clots or sediment. The current procedure for administering CBI is as follows: The health care provider first inserts a triple lumen catheter into the patient's bladder. The triple lumen catheter is composed of one port to inflate the balloon on the end of the catheter, another port for irrigation fluid to flow into the bladder, and a third port for fluid draining from the bladder. While the catheter is in the bladder, the patient's bladder emptying relies upon a free flowing and patient outflow port. If the outflow port becomes obstructed, especially during CBI, the bladder will become distended, and acute urinary retention will occur. This acute urinary retention can have serious immediate and long term consequences including, but not limited to, pain, infection, urosepsis, kidney failure, bladder rupture, hydronephrosis, gross hematuria, and exacerbation of underlying medical conditions.

In previously known irrigation systems, irrigation solution is contained within a bag mounted on an IV pole. The irrigation solution flows by gravity through IV tubes connected to a three-way catheter setup. The three-way catheter setup allows the irrigation fluid to enter the bladder through one lumen, flush the bladder, and then empty into a collection system through an outflow lumen in the catheter.

The health care provider hangs a full or partially full irrigation fluid bag from an irrigation pole. The output from the irrigation bag is connected via irrigation tubing to the second lumen on the catheter. The third lumen on the catheter is connected to the tubing on the urine meter bag suspended from the patient's bed. The health care provider typically adjusts the irrigation flow via a titration wheel located between the irrigation fluid bag and the catheter. The health care provider titrates the irrigation fluid flow rate to keep the fluid leaving the catheter and filling the urine meter bag a clear to light pink color. This color generally corresponds to a low blood component in the fluid. The lower the blood component in the fluid the lower the probability of clots forming that could obstruct the system.

The flow rate can vary widely from a slow drip rate to high volume stream. The high flow rates are physically not attainable with intravenous tubing as the diameter is too small. Bladder irrigation tubing is a much larger diameter tubing specifically designed for a wide range of fluid flow rates. The health care provider then leaves the patient's room and continues with other duties not pertaining to the CBI procedure.

Periodically, the health care provider checks on the patient and the state of the CBI procedure. The increments between checks are based on experience monitoring similar procedures or guessing when the irrigation fluid bag will be empty based on the observed titration rate. Without accurate knowledge of when the irrigation fluid bag will run out, many health care providers either check on the patient too frequently, thus diminishing their overall efficiency, or check too infrequently, thus putting the patient at risk for complications. When it is observed that an issue exists with the CBI procedure, the health care provider performs various actions to correct the issue with the procedure.

Typical observed, visual or otherwise, issues with the CBI procedure are a total blockage of the catheter and an empty irrigation fluid bag. A total blockage of the catheter outflow port can cause the bladder to fill to a level which causes the patient severe pain and, in certain cases, a rupture of the bladder which can result in additional surgeries, urinary tract infections, prolonged CBI, and increased hospital length of stay. Many times, a complete blockage can be articulated by the patient to the health care provider who can then irrigate the catheter and, thus, resolve the issue. However, with very elderly or demented patients who are unable to communicate effectively with the health care provider and, thus, unable to articulate the discomfort indicative of a completely blocked catheter, the condition is largely missed by the health care provider and severe pain, and, in certain cases, a rupture in the bladder will result. These circumstances can cause urinary tract infections, additional surgeries, increased hospital length of stay, and the need for further CBI. All of these complications increase patient's morbidity and mortality.

An empty irrigation fluid bag causes the continuous bladder irrigation procedure to become non-continuous or intermittent and, thus, potentially results in unnecessary complications for the patient. Ideally, when an irrigation fluid bag is near empty, the health care provider should be preparing to hang a new irrigation fluid bag to keep the irrigation fluid flow continuous. Sometimes fluid bags are connected in tandem via Y-tubing to help facilitate the continuous nature of CBI. Many times, if not the majority, the irrigation fluid bag will inadvertently be allowed to run out. Once the irrigation fluid bag has run out, the bladder is no longer being continuously irrigated and blood clots can start to form in the bladder and the urinary tract. These clots can cause the need for further CBI procedures, urinary tract infections, additional surgeries, and increased hospital length of stay.

While the above two issues are easily observed by the experienced health care provider, a partial blockage of the outflow port of the catheter is not easily observed, even by the most experienced health care providers. It is the precursor to a complete obstruction. A partial blockage of the catheter can cause gradually increasing discomfort that can often be overlooked by both the health care provider and the patient. This increased amount of retained urine and irrigant can have significant consequences similar to those previously listed. It will also leave a smaller functional bladder capacity. With regards to CBI, this will mean that, when a partial blockage is converted to a complete blockage, it will be a shorter time period until the bladder reaches maximum capacity as urine and irrigation fluid flow into the bladder, thus, increasing the risk for serious complications.

Regardless of these longstanding issues with Continuous Bladder Irrigation, for over 60 years, this procedure has remained the standard of care in hospitals around the world for management of gross hematuria. The aforementioned difficulties encountered routinely by health care providers managing CBI are universally recognized. The cost incurred by the health care system over that time period in managing these complications that are addressed by this method and device are astronomical.

### SUMMARY OF THE INVENTION

The present invention alerts the user when the gravity irrigation rate of irrigation fluid decreases by a set amount or the irrigation fluid remaining in the irrigation fluid bag approaches a critical level, both of which can result in blood clots obstructing the fluid outflow from the bladder and cause increased morbidity and mortality in patients. The specifics of both events are intrinsic only to continuous bladder irrigation, not to intermittent gravity irrigation monitoring devices or intravenous infusion monitoring. The definition of infusion in medical dictionaries excludes irrigation of a cavity or hollow organ. Infusion implies a fluid or medicine is used to treat tissues or the whole body. CBI, a pure irrigation system, is essentially washing out the inside of the bladder. The fluid is not absorbed during CBI as the bladder lining is impermeable to diffusion and osmosis.

The presently disclosed invention monitors the time remaining during continuous bladder irrigation and alerts medical personnel if the time remaining changes. The monitor measures the mass of a bag of solution suspended from an IV pole and sounds an alarm or otherwise alerts medical personnel in the event of a change in the flow time remaining. The output flow rate can also be monitored by a similar device to alert personnel that inflow of solution is exceeding outflow of solution. It is able to display the time remaining until completion of the hanging irrigant solution. It is also able to detect partial or complete obstruction by sensing a particular change in the flow rate. Once the titration rate is set by the nursing personnel, the flow rate curve of the fluid draining to gravity is predictable. This predictability is what enables the devices to detect a clinically significant change in that flow rate curve that is reflected as a change in the time remaining. This would prompt an “event,” thus alerting the nursing personnel. If an obstruction occurred and caused reflux of fluid back up into the system retrograde, the bag mass would increase. This would be noted as an event based on the change in the flow rate curve, thus alerting the nursing personnel. The CBI Alarm (CBIA) can handle the largest irrigant bags used in routine medical care, up to 5 Liters. It could be adjusted for higher capacity bags. The device is envisioned to potentially send information to a nurse or nursing station via wireless connection.

### BRIEF DESCRIPTION OF THE DRAWINGS

Further advantages of the invention will become apparent by reference to the detailed description of preferred embodiments when considered in conjunction with the drawings:

FIG. 1 depicts an embodiment of the claimed device as attached to an IV pole.

FIG. 2 depicts the claimed device attached to an IV pole.

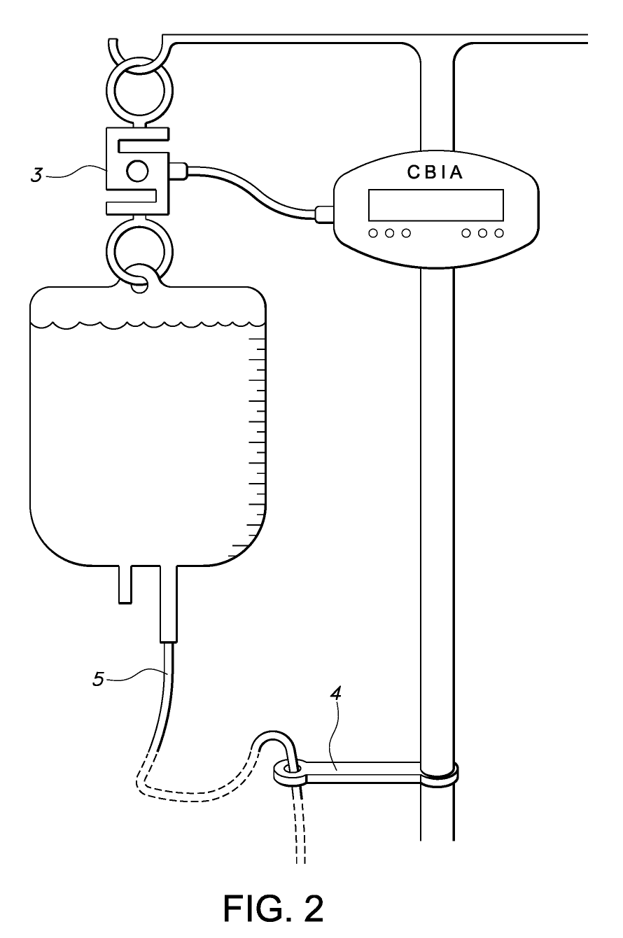

FIG. 3 depicts various embodiments of the claimed device.

FIG. 4 depicts a front view of another embodiment of the claimed device that incorporates an IV pole.

FIG. 5 depicts a side view of another embodiment of the claimed device that incorporates an IV pole.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

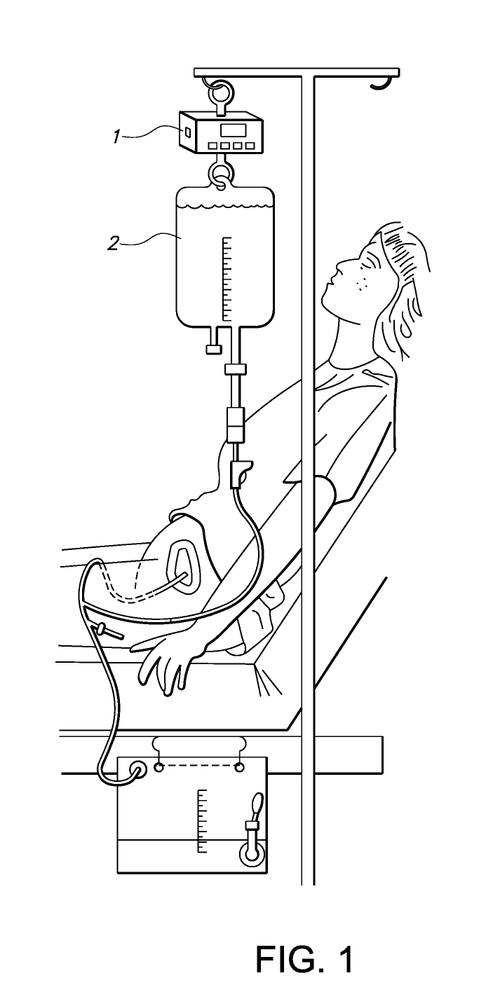

Referring to the drawings, FIG. 1. illustrates an embodiment of the claimed device, also referred to herein as the Continuous Bladder Irrigation Alarm, or CBIA 1, in use with a patient. CBIA 1 is a monitoring device for a group of procedures collectively known as CBI or continuous bladder irrigation. These procedures require the bladder of a human being to be continuously irrigated using a sterile normal saline, glycine, sterile water, or other sterile solution administered via a gravity irrigation system. The CBIA device is comprised of analog and digital electrical components necessary to perform functions during a CBI procedure as described below.

As will be appreciated from the drawing, a continuous bladder irrigation system of the prior art can be used in conjunction with the presently claimed device with the exception that the CBIA 1 is interposed between the IV pole and the irrigation bag 2. Thus, the mass of the irrigation bag 2, along with drip catheter bag and tubing, exerts a downward force against the CBIA 1. The CBIA 1 is designed to measure this force by using a strain gage load cell 3 or other load cell that provides an electrical signal in response to a force.

In a preferred embodiment, the CBIA 1 comprises of a strain gauge load cell 3, a display, input buttons, circuitry for calculating the flow rate of irrigation solution, and means for alerting medical personnel, such as an audible alarm or visible indicator, or connection to a paging system. The circuitry and display are contained within a housing, preferably made of plastic or metal, or other non-porous surface. The strain gage load cell 3 can either be located within the CBIA 1, or, optionally, it may be located at a distance from the CBIA 1. In one embodiment, the strain gauge load cell 3 is attached to the CBIA 1 by means of a USB cable. However, it is contemplated that wireless communication means may also be used between the strain gage load cell 3 and the CBIA 1. Hooks are used to attach the strain gage load cell 3 or CBIA 1 to the IV pole.

Various CBIAs can be designed to operate using either AC or DC power. A CBIA that relies only on AC power would prevent a patient from being wheeled into another room. In a preferred embodiment, the CBIA includes a rechargeable battery so that it can continue to operate in the event of a loss of power. A single-use disposable model that uses batteries could also be implemented.

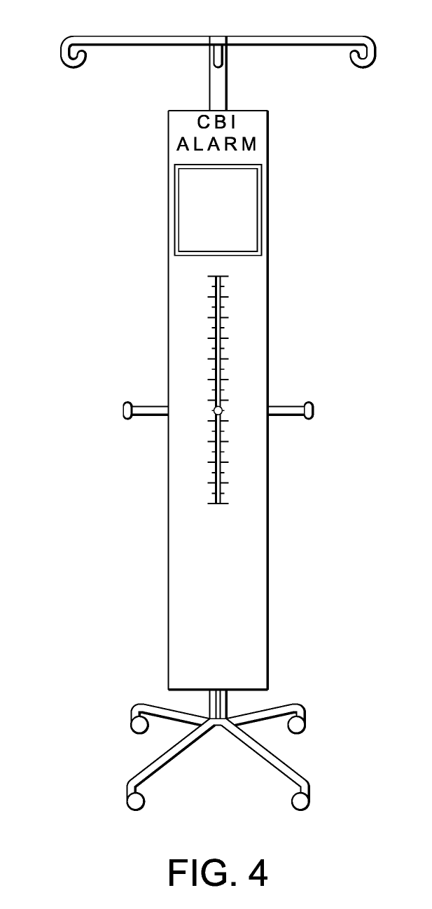

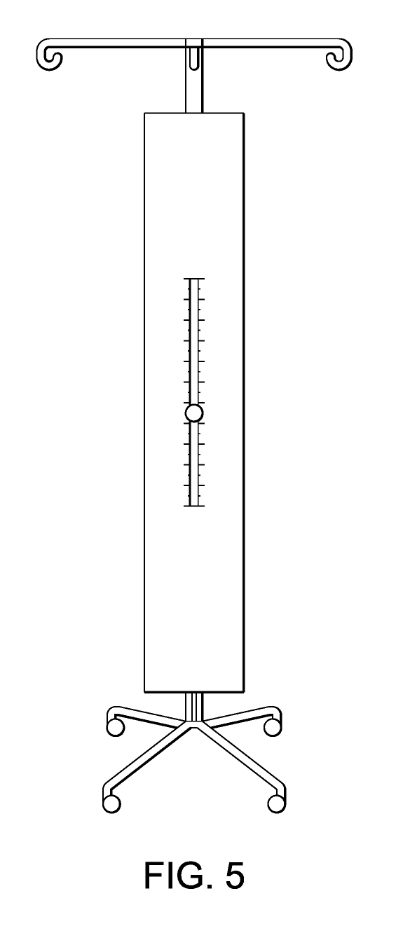

In another embodiment, illustrated in FIG. 4 and FIG. 5, the CBIA 1 and IV pole can be built as a single unit. Preferably, this free standing single unit will also have wheels so it can be rolled into different locations. It is contemplated that this unit would have a touch screen display for ease of use, and also incorporate a backup battery supply.

Operation

As can be appreciated from FIG. 1, the CBIA 1 can be attached to an IV pole, with the irrigation bag 2 suspended directly from the CBIA 1. The medical personnel that is preparing for the continuous bladder irrigation procedure could then input the initial volume of the irrigation solution into the CBIA 1 using the buttons on the CBIA 1. Some commonly used values could be preset into the CBIA 1, and other nonstandard values could be input manually. However, based on the preferred embodiment, no values would need to be input as the design of the CBIA 1 will work with any fluid load.

The irrigation bag 2 may be further stabilized by tubing stabilizer 4. Tubing stabilizer 4 is a device that holds a portion of the CBIA tubing 5 so that the reading from strain gauge load cell 3 is not heavily affected by slight patient motion.

Circuitry within the CBIA 1 has the capability of ascertaining the original mass of the irrigation bag 2, and it can also be zeroed out to reset the volume. The circuitry also allows the CBIA 1 to regularly monitor the mass of the irrigation bag 2. If the irrigation solution is properly flowing, the mass of the bag should gradually and steadily decrease. Based on the decrease in mass of the irrigation bag 2, the CBIA 1 can calculate the flow time remaining of the irrigation solution.

When the CBIA 1 detects an increased resistance, the time remaining changes to a longer interval, thus, it goes into an alarm state and alerts medical personnel. In a preferred embodiment, the medical personnel is alerted to a small reduction (+/−25% to +/−50%) in the flow of the irrigation fluid from the irrigation fluid bag. This irrigation fluid flow characteristic is also inherent only to the CBI procedure and is indicative of a blood clot or debris causing a partial blockage of the outflow port of the catheter. It is also indicative of a condition that could cause morbidity or mortality to a patient if left unchecked.

The alerts may be audible, visual, or even wireless to a pager or other handheld module. The increase in flow time remaining could indicate that the irrigation tubing has become crimped, clot retention has occurred or the irrigation bag 2 has emptied and needs to be replaced.

One claimed aspect of this invention is that the CBIA 1 can also estimate the completion time of any irrigation bag based on the initial volume and flowrate. The estimated completion time can be updated by the CBIA 1 at regular intervals so that it accounts for subclinical changes in flowrate. If clinically significant flowrate changes occur, this would indicate an event, thus prompting an alert to the nursing personnel. As the irrigation bag approaches completion, the estimated completion time will be more accurate. When the CBIA 1 time remaining is near or at zero, it can then alert medical personnel so that a nurse can replace the bag without delay.

In a preferred embodiment, the user is informed of the calculated time remaining before the CBI irrigation fluid in the irrigation fluid bag reaches a preset level that necessitates changing the bag; or the user enters the current local time into the system, and the device informs the user of the actual time of day or night when the irrigation will be approaching the critical level.

The CBI procedure requires that the irrigation fluid bag has to be changed out prior to the irrigation fluid running out so that blood clots and sediment do not consolidate during a low flow or no flow state and result in obstruction. The minimum safe irrigation fluid level of an irrigation fluid bag is a characteristic that is inherent only to the CBI procedure.

Ignorance of the time remaining before the CBI fluid in the irrigation fluid bag reaches a preset level, and, thus, the irrigation fluid bag running out of irrigation fluid, could result in morbidity or mortality of a patient. Ignorance of the time remaining before the CBI fluid in the irrigation fluid bag reaches a preset level will result in inefficiencies of the health care provider. Conversely, knowledge of the time remaining of CBI fluid will make the health care provider more efficient.

In another embodiment, the user is alerted to the CBI irrigation fluid in the irrigation fluid bag reaching a preset level that necessitates changing the bag. The CBI procedure requires that the irrigation fluid bag has to be changed out prior to the irrigation fluid running out so that blood clots and sediment do not consolidate during a low flow or no flow state and result in obstruction. The minimum safe irrigation fluid level of an irrigation fluid bag is a characteristic that is inherent only to the CBI procedure. The minimum safe irrigation fluid flow time remaining of an irrigation fluid bag is a characteristic that is inherent only to the CBI procedure.

Ignorance of the irrigation fluid level in the irrigation fluid bag reaching a minimal safe level or minimum safe flow time remaining could potentially allow the irrigation fluid bag to run out of irrigation fluid resulting in morbidity or mortality of a patient.

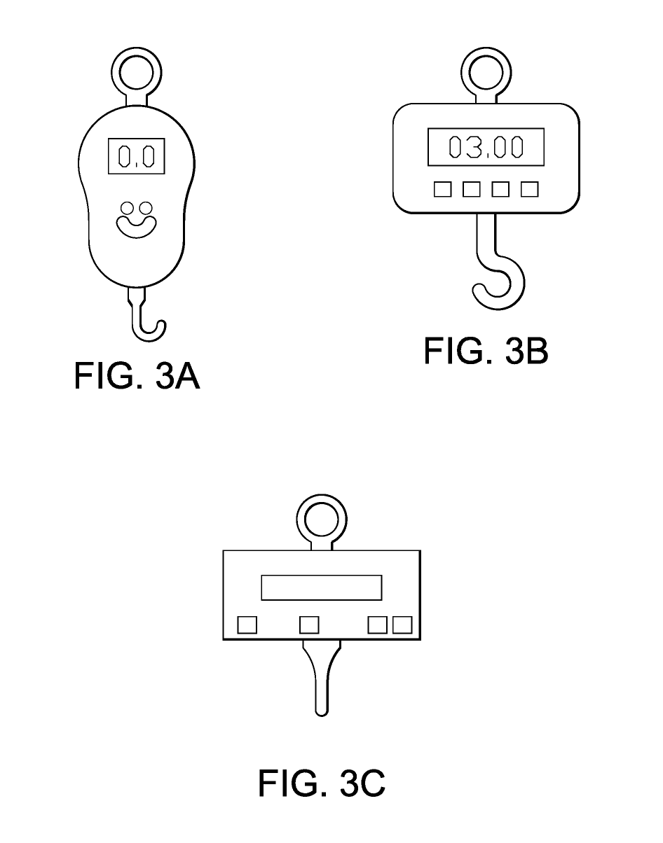

As will be appreciated from FIG. 3, the CBIA 1 is preferably small and ergonomic so that it can be handheld; however, it can be any size or shape. In fact, in another embodiment, the strain gauge load cell 3 or CBIA 1 containing a strain gauge load cell 3 is built directly into an IV pole.

It is able to detect and/or filter artifactual data caused by coughing, breathing, moving, talking, bladder spasms, etc., potentially incorporating urodynamic leads to more precisely measure this data.

Usage Example

Patient A comes to the recovery room after a transurethral resection of the prostate (TURP) and has a 3-way Foley catheter for continuous bladder irrigation. The CBIA 1 is placed on the IV stand, and a 5-liter bag of normal saline is suspended from it. The nurse presses the start button. A short time after the irrigation begins, the CBIA 1 displays 1 hour remaining. Five minutes later, the display reads 55 minutes remaining. Subsequently, the irrigant time remaining changes to 2 hours prompting an alert that an event has occurred. This suggests increased resistance in the system, likely a clot. The nurse assesses and manually irrigates a clot out of the bladder. The irrigation solution continues to flow normally until about 50 minutes later when the alarm sounds to indicate that the bag is almost empty or at time remaining of zero. A new irrigation bag is subsequently placed on CBIA 1, and the CBIA 1 is reset.

Alternative Embodiments.

Several variations in the device have been contemplated. In one embodiment, the CBIA 1 is envisioned to potentially have controls to automatically stop the gravity infusion in the event of an obstructed system by mechanically interrupting the flow or by lowering the infusion bag to lower the hydrostatic pressure head. It may also have the potential to send signals to have the Foley catheter automatically irrigated in the event of an occlusion.

In other embodiments, the flow rate can be displayed based on change in mass/time. Also the device may be a single-use device that is used once and then discarded or recycled. For example, it can be built into an irrigation bag.

In yet another embodiment, the device can alert the user when the Foley catheter bag is full.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We claim:

1. A device for detecting the formation of a clot during a continuous bladder irrigation comprising:
a. an irrigation bag support capable of detecting changes in mass in the irrigation bag;
b. circuitry for computing the flowrate of irrigation wherein said flowrate is calculated as a change in mass in the irrigation bag over time.

2. The device of claim 1 further comprising an alert system that signals an alarm when there is a change in flowrate.

3. The device of claim 2 wherein said alert is audible.

4. The device of claim 2 wherein said alert occurs by electronic communication.

5. A device for determining the completion of an irrigation bag during a continuous bladder irrigation comprising:
a. an irrigation bag support capable of detecting changes in mass in the irrigation bag;
b. circuitry for computing the flowrate of irrigation wherein said flowrate is calculated as a change in mass in the irrigation bag over time.

6. The device of claim 5 further comprising an alert system that signals an alarm when there is a change in flowrate.

7. The device of claim 5 wherein said alert is audible.

8. The device of claim 5 wherein said alert occurs by electronic communication.

9. The device of claim 5 further comprising an IV pole for holding said irrigation bag.
